SEQUENCE LISTING (4 sequences)

SEQ ID NO: 1 - DNA, 1215 bp, Zea mays
gaattcaccggcgcataatcattcatgcccatccatctctatctctgtgggtcgtcgttgtgtgtaatttgagtacgcgcgtacacgtactgcttttgtttcatttgagcactgttcgtgtaagcgtcgagttgccctgccatcgtctctctctctctcgatctctactatttctgtgtgtgcgcgcttctatttttccaatgttgagttacgcatatttgtccagtaaccttgttttgcttaatcacacacagctcccaaattccagtcgttctgtggtgacggttggttgtggggtgtggcggcgacgtcagtcatgggagaagagatccgaatgcatgggggtgttggcgctttctctgtccctctccctctccctgtcttctcgcgcgctcgccgtcgtcatggctcgacgaacagacattcactgccttccccgcgcgcttcagacagaccacgcttgtacgtagcgccgcttctctagtcgccggagcaggcacgagtgggtggcgaagaggccacggcccgacctgctagcgccagcgaccgcatctcgccaccagccgctgcccgctggccgctgccaccgtatgttgggttgggctgaagcctgaagcccgtgacctttttctttttctttattctttccttttcttttgccggacaagacaagtgatgttgcaaaatccatataaaatcctagagaaactataaagtttttttttgttgaactccttataacaacgtctaccctttaatatatggtatataatttcacatgttttagtataaattgtttactgtatttctatgtctatatgctcttggtaagattgtttataaattttaaatttaaaatcttaaaagtcataaacttatagaaaaaaattaggatcctaaacaattttaaacaaaaaaaaaaacttttcaattacaaaatcgtatgtgttctttatgcatgcatgcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgtagcatacactagtgcttttgctagctacttgtgcgagaagagagagcgcacggcgatagatatg

SEQ ID NO: 2 - DNA, 531 bp, Zea mays
cgatccagatcaagcagtactcgactaccgccactacactacctgctctgctcgtgtcttgtttccagttttctttctctgtaagcggtgtagggtttgccaaacttttatgtacgaattatgaatatcaataaagtcgatctgctgctgcgtttcttggcaccaacaatctttccgcctcatcagagcaactcccccataaatttaaatttcctatagtgaatttgctagtctgctagcgacttactaagtagttcatactgaacgggagaacaagtgccatcgaggcttcatgcacatttcactgaccataaacaacacagtggtaaaaaaccatcccatcgaggcaaggcaaagatcctgagttcaacatacctctctgtaaaaatgcaggggtaaaacttgttatttcttttcttgaaaaccttgtctgtcatataactaattcaaaattaggaaaacaaaaaagagaggaaggaatgctaccggctcaaacctcgatagacaaacagcaactagaattactacaga

SEQ ID NO: 3 - DNA, 390 bp, Zea mays
atggcgcatagcaagcgcgaaatcaagcccgggagggcgcataccaaggtggccggcgacgacgagatgctgcggaccgggttcttcgacggcacgccgctggagggcggcaagatcgccgactcccagcccgtcgacctcttcgccgctgcccgccgcgtcgccgacgccccgtactcatcgcagcaggaaggaccacacgaggaggggaccaacaagaacgccgtcatcgcggagtcggagcccgtcgacctgcccgcgagcgcgcggggcgtggcggaggtgaaccgtgctgggccggaggagcagacaggccgcggtcggcagggcatggccgagcccaccgccggtggacgtcgcttgggattaggtcgcccggccccggcttaa

SEQ ID NO: 4 - DNA, 467 bp, Zea mays
taccctttaatatatggtatataatttcacatgttttagtataaattgtttactgtatttctatgtctatatgctcttggtaagattgtttataaattttaaatttaaaatcttaaaagtcataaacttatagaaaaaaattaggatcctaaacaattttaaacaaaaaaaaaaacttttcaattacaaaatcgtatgtgttctttatgcatgcatgcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgtagcatacactagtgcttttgctagctacttgtgcgagaagagagagcgcacggcgatagatatg

(19) United States
(12) Patent Application Publication
(10) Pub. No.: US 2009/0007302 A1
(43) Pub. Date: Jan. 1, 2009

(54) SEED-PREFERRED REGULATORY ELEMENT

(75) Inventor: Shane ABBITT, Ankeny, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 12/164,528

(22) Filed: Jun. 30, 2008

Related U.S. Application Data
(60) Provisional application No. 60/946,758, filed on Jun. 28, 2007.

(51) Int. Cl. A01H 5/00; C07H 21/04; C12N 15/82; C12N 5/10

(52) U.S. Cl. 800/312; 536/24.1; 435/320.1; 435/419; 800/295; 800/317.2; 800/317.4; 800/320.1; 800/320.2; 800/320.3; 800/314; 435/455

(57) ABSTRACT

The present invention provides compositions and methods for regulating expression of nucleotide sequences of interest in a plant. Compositions are novel nucleotide sequences for a seed-preferred promoter and terminator associated with the maize embryo abundant protein 2 coding region. A method for expressing a nucleotide sequence of interest in a plant using the regulatory sequence disclosed herein is provided.

### CROSS-REFERENCE TO RELATED APPLICATIONS

This application claims priority under 35 U.S.C. §120 to provisional application Ser. No. 60/946,758 filed Jun. 28, 2007 herein incorporated by reference in its entirety.

### FIELD OF THE INVENTION

The present invention relates to the field of plant molecular biology, more particularly to regulation of gene expression in plants.

### BACKGROUND OF THE INVENTION

Expression of DNA sequences in a plant host is dependent upon the presence of operably linked regulatory elements that are functional within the plant host. Choice of the regulatory element will determine when and where within the organism the DNA sequence is expressed. Where continuous expression is desired throughout the cells of a plant, and/or throughout development, constitutive promoters are utilized. In contrast, where gene expression in response to a stimulus is desired, inducible promoters are the regulatory element of choice. Where expression in specific tissues or organs are desired, tissue-preferred promoters may be used. That is, they may drive expression in specific tissues or organs. Such tissue-preferred promoters may be temporal, constitutive, or inducible. In either case, additional regulatory sequences upstream and/or downstream from a core promoter sequence may be included in expression constructs to bring about varying levels of expression of nucleotide sequences in a transgenic plant.

As this field develops and more genes become accessible, a greater need exists for transformed plants with multiple genes. These multiple exogenous genes typically need to be controlled by separate regulatory sequences, however. Further, some genes should be regulated constitutively whereas other genes should be expressed at certain developmental stages and/or locations in the transgenic organism. Accordingly, a variety of regulatory sequences having diverse effects is needed.

Diverse regulatory sequences are also needed, as undesirable biochemical interactions can result from using the same regulatory sequence to control more than one gene. For example, transformation with multiple copies of a regulatory element may cause problems, such that expression of one or more genes may be affected.

Isolation and characterization of seed-preferred promoters and terminators that can serve as regulatory elements for expression of isolated nucleotide sequences of interest in a seed-preferred manner are needed for impacting various traits in plants and in use with scorable markers. The inventor has isolated just such a promoter.

### BRIEF SUMMARY OF THE INVENTION

The present invention therefore relates to an isolated regulatory sequence that regulates transcription in a seed embryo-preferred manner. Such regulatory sequence is preferably a sequence natively associated with, and that drives expression in, the coding regions of eap2 (embryo abundant protein 2).

The present invention further relates to recombinant expression cassettes comprising such a regulatory sequence operably linked to a nucleic acid of interest, or a vector comprising such an expression cassette.

The present invention also relates to plant cells having stably incorporated in its genome such a regulatory sequence. Such plant cells may be monocots or dicots, and could include maize, wheat, rice, barley, sorghum, millet, rye, soybeans, alfalfa, oilseed Brassica, cotton, sunflower, potatoes, or tomatoes. The present invention also includes plants stably transformed with such an isolated regulatory sequence, and transgenic seed obtained from such plants.

The present invention also relates to a method for modulating expression of a nucleic acid of interest in a plant, comprising introducing into a plant cell or tissue a polynucleotide molecule comprising one of the above-described regulatory sequences operably linked to a nucleic acid of interest, regenerating a plant from the plant cell wherein the plant expresses the nucleic acid of interest.

Additional detail regarding the present invention will become evident from the further description provided.

### BRIEF DESCRIPTION OF THE DRAWINGS

FIG. 1 shows the summary of LYNX™ data illustrating the native expression of embryo abundant protein 2 in zea mays.

FIGS. 2 and 3 show photographs of the expression of the DS-RED marker in an experimental group using the ZM-EAP2 promoter.

FIG. 4 shows photographs of the expression of the DS-RED marker in an experimental group using the ZM-EAP2B truncated promoter.

### DETAILED DESCRIPTION

In accordance with the invention, nucleotide sequences are provided that allow regulation of transcription in seed tissue. The sequences comprise regulatory elements associated with seed formation and seed tissues. Thus, the compositions of the present invention comprise novel nucleotide sequences for plant regulatory elements natively associated with the nucleotide sequences coding for embryo abundant protein 2, herein identified as EAP2.

In an embodiment, the regulatory element drives transcription in a seed-preferred manner, wherein said regulatory element comprises a nucleotide sequence selected from the group consisting of: a) sequences natively associated with, and that regulate expression of DNA coding for maize EAP2 (embryo abundant protein 2; b) the nucleotide sequence set forth in SEQ ID NO: 1; or c) a sequence comprising a fragment of the nucleotide sequence set forth in either of SEQ ID NO: 1.

In another embodiment of the invention the regulatory element comprises bases 749 to 1215 of SEQ ID NO: 1 (“first truncation,” SEQ ID NO: 4).

Further embodiments are to expression cassettes, transformation vectors, plants, plant cells and plant seed comprising the above nucleotide sequences. The invention is further to methods of using the sequence in plants and plant cells. An embodiment of the invention further comprises the nucleotide sequences described above comprising a detectable marker.

During the reproduction process, angiosperms produce an ovary, which, together with its seed develop into a fruit, that is, a ripened ovary or ovaries, and adjacent parts that may be fused to it. The mature ovary wall is the seed and encloses the seeds. Manipulation of seed properties, expressing proteins to the seed, and expressing markers to the seed has numerous uses in the plant industry. A promoter expressing proteins in the seed layer is valuable for a variety of applications in expressing proteins including controlled expression in seed tissue of such proteins.

Such a promoter is also useful to target sequences encoding proteins for disease resistance to the seed. Additionally, linking a promoter which preferentially expresses to the seed with a marker, and, in particular, a visual marker, is useful in tracking the expression of a linked gene of interest.

A method for expressing an isolated nucleotide sequence in a plant using the regulatory sequences disclosed herein is provided. The method comprises transforming a plant cell with an isolated nucleotide sequence operably linked to one or more of the plant regulatory sequences of the present invention and regenerating a stably transformed plant from the transformed plant cell. In this manner, the regulatory sequences are useful for controlling the expression of endogenous as well as exogenous products in a seed-preferred manner.

Frequently it is desirable to have preferential expression of a DNA sequence in a tissue of an organism. For example, increased resistance of a plant to insect attack might be accomplished by genetic manipulation of the plant's genome to comprise a tissue-preferred promoter operably linked to an insecticide gene such that the insect-deterring substances are specifically expressed in the susceptible plant tissues. Preferential expression of the nucleotide sequence in the appropriate tissue reduces the drain on the plant's resources that occurs when a constitutive promoter initiates transcription of a nucleotide sequence throughout the cells of the plant.

Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that tissue-preferred expression of the antisense sequence produces an RNA transcript that interferes with translation of the mRNA of the native DNA sequence in a subset of the plant's cells.

Under the regulation of the seed-preferred regulatory elements will be a sequence of interest, which will provide for modification of the phenotype of the seed. Such modification includes modulating the production of an endogenous product, as to amount, relative distribution, or the like, or production of an exogenous expression product to provide for a novel function or product in the seed.

### Definitions

By “embryo-preferred” is intended favored spatial expression in the embryo of the seed.

By “isolated” is intended material, such as a nucleic acid or protein, which is: (1) substantially or essentially free from components which normally accompany or interact with the material as found in its naturally occurring environment or (2) if the material is in its natural environment, the material has been altered by deliberate human intervention to a composition and/or placed at a locus in a cell other than the locus native to the material.

By “nucleic acids providing improved output traits” is intended genes that confer or contribute to an altered plant characteristic.

By “nucleic acids providing improved agronomic traits” is intended genes that confer or contribute to one or more agronomic traits.

By “ovary” is meant the ripened ovary or ovaries, and adjacent parts that may be fused to it.

By “promoter” is intended a regulatory region of DNA capable of regulating the transcription of a sequence linked thereto. It usually comprises a TATA box capable of directing RNA polymerase II to initiate RNA synthesis at the appropriate transcription initiation site for a particular coding sequence.

By “regulatory element” is intended sequences responsible for the expression of the associated transcript including, but not limited to, promoters, terminators, enhancers, introns, and the like.

By “seed-preferred” is intended favored expression in the seed, the wall of the ovary of a plant, and the like.

By “terminator” is intended sequences that are needed for termination of transcription: a regulatory region of DNA that causes RNA polymerase to disassociate from DNA, causing termination of transcription.

A promoter may additionally comprise other recognition sequences generally positioned upstream or 5′ to the TATA box, referred to as upstream promoter elements, which influence the transcription initiation rate and further include elements which impact spatial and temporal expression of the linked nucleotide sequence. It is recognized that having identified the nucleotide sequences for the promoter region disclosed herein, it is within the state of the art to isolate and identify further regulatory elements in the 5′ region upstream from the particular promoter region identified herein. Thus the promoter region disclosed herein may comprise upstream regulatory elements such as those responsible for tissue and temporal expression of the coding sequence, and may include enhancers, the DNA response element for a transcriptional regulatory protein, ribosomoal binding sites, transcriptional start and stop sequences, translational start and stop sequences, activator sequences and the like.

In the same manner, the promoter elements which enable expression in the desired tissue such as the seed can be identified, isolated, and used with other core promoters to confirm seed-preferred expression. By core promoter is meant the minimal sequence required to initiate transcription, such as the sequence called the TATA box which is common to promoters in genes encoding proteins. Thus the upstream region of EAP2 can optionally be used in conjunction with its own or core promoters from other sources. The promoter may be native or non-native to the cell in which it is found.

The isolated promoter sequence of the present invention can be modified to provide for a range of expression levels of the isolated nucleotide sequence. Less than the entire promoter region can be utilized and the ability to drive seed-preferred expression retained. It is recognized that expression levels of mRNA can be modulated with specific deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. Generally, at least about 20 nucleotides of an isolated promoter sequence will be used to drive expression of a nucleotide sequence.

It is recognized that to increase transcription levels enhancers can be utilized in combination with the promoter regions of the invention. Enhancers are nucleotide sequences that act to increase the expression of a promoter region. Enhancers are known in the art and include the SV40 enhancer region, the 35S enhancer element, and the like.

The promoter of the present invention can be isolated from the 5′ region of its native coding region or 5′ untranslated region (5′ UTR). Likewise the terminator can be isolated from the 3′ region flanking its respective stop codon. Methods for isolation of promoter regions are well known in the art.

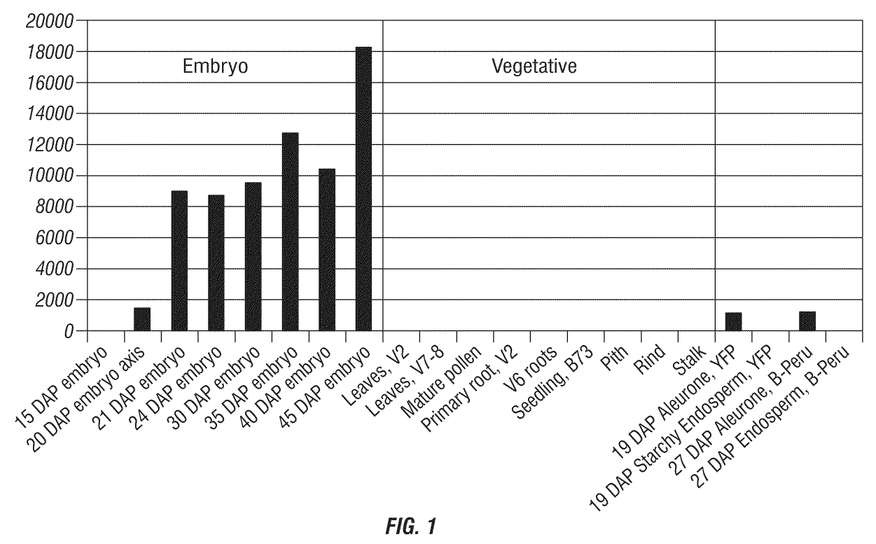

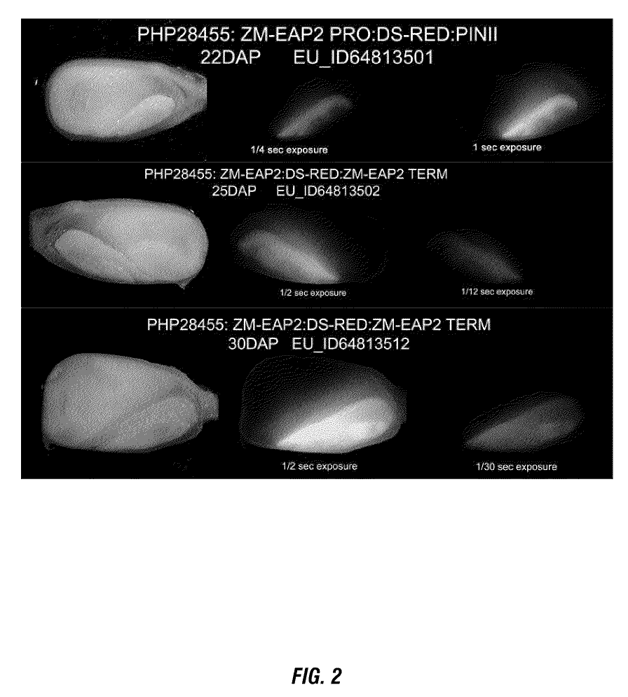

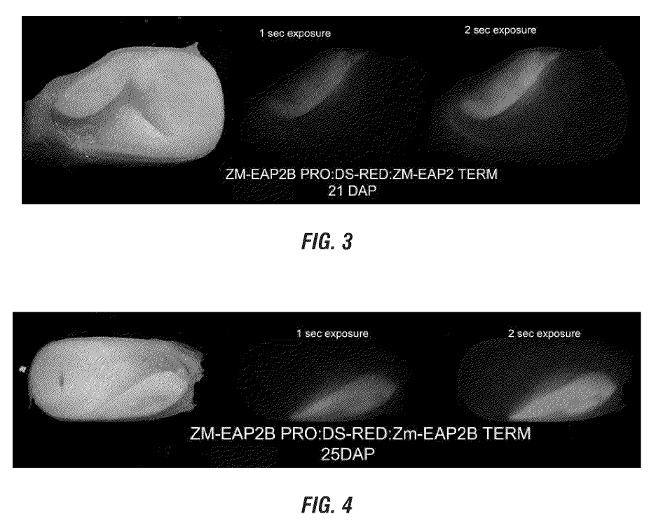

The ZM-EAP2 promoter set forth in SEQ ID NO:1 is 1215 nucleotides in length. The ZM-EAP2 promoter was isolated from the Zea mays EAP2 coding region and the ZM-EAP2 transcript is set forth as SEQ ID NO:3, and the terminator region is SEQ ID NO:2. It was isolated based on MPSS (Massively Parallel Signature Sequencing) technology from LYNX™ (see Brenner et al, Nature Biotechnology 18:630-634 (2000)) expression analysis showing strong expression in 20-40 DAP (days after pollination) maize seed. The results of the native expression analysis can be found in FIG. 1. The results of transgenic expression analysis for the ZM-EAP2 promoter are illustrated in FIGS. 2 and 3. The results of transgenic expression analysis for the ZM-EAP2B promoter truncation are illustrated in FIG. 4. The ZM-EAP2 promoter and the ZM-EAP2B promoter truncation can address expression problems by providing this pattern of expression.

Motifs of about six or eight bases within the ZM-EAP2 promoter sequence were discovered by searching for sequences of similar size and within 100 bases of the position in which they were located. The following motifs are found in the ZM-EAP2 promoter as represented in Table 1 below.

TABLE 1

| EAP2 Motif/ | | | |
|---|---|---|---|
| Deletion | Known Reg | | |
| Number | Element | Name | Description |
| CAAT | CAAT | CAATBOX1 | Common promoter motif found |
| | | | ~200 bp upstream of transcriptional |
| | | | start; signals binding of RNA |
| | | | transcription factor. |
| G-Box | CACGTC | ABREZMREB28 | cis-acting sequence present in |
| | | | several plant promoters |
| RY Repeat | CATGCATG | RYREPEAT4 | quantitative seed expression; Gene: |
| | | | Vicia faba LeB4; Soybean glycinin |
| | | | (Gy2); other dicot and monocot |
| | | | seed protein genes; transacting |
| | | | factor: unknown; “Sph box” found |
| | | | in rice (O.s.) Osem gene promoter; |
| | | | enhances the activation by VP1; |
| | | | Binding site of Arabidopsis B3- |
| | | | domain-containing transcription |
| | | | factor FUS3; TRAB1, bZIP |
| | | | transcription factor, interacts with |
| | | | VP1 and mediates abscisic acid- |
| | | | induced transcription |
| CCAAT Box | CCAAT | CCAATBOX1 | MYBHv1 Binding site |

The promoter regions of the invention may be isolated from any plant, including, but not limited to corn (Zea mays), oilseed Brassica (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), millet (Panicum spp.), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats (Avena sativa), barley (Hordeum vulgare), vegetables, ornamentals, and conifers. Preferably, plants include corn, soybean, sunflower, safflower, oilseed Brassica, wheat, millet, barley, rye, rice, alfalfa, and sorghum.

Promoter sequences from other plants may be isolated according to well-known techniques based on their sequence homology to the homologous coding region of the coding sequences set forth herein. In these techniques, all or part of the known coding sequence is used as a probe which selectively hybridizes to other sequences present in a population of cloned genomic DNA fragments (i.e. genomic libraries) from a chosen organism. Methods are readily available in the art for the hybridization of nucleic acid sequences. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., eds., Greene Publishing and Wiley-Interscience, New York (1995).

“Functional variants” of the regulatory sequences are also encompassed by the compositions of the present invention. Functional variants include, for example, the native regulatory sequences of the invention having one or more nucleotide substitutions, deletions or insertions. Functional variants of the invention may be created by site-directed mutagenesis, induced mutation, or may occur as allelic variants (polymorphisms).

As used herein, a “functional fragment” is a regulatory sequence variant formed by one or more deletions from a larger regulatory element. For example, the 5′ portion of a promoter up to the TATA box near the transcription start site can be deleted without abolishing promoter activity, as described by Opsahl-Sorteberg, H-G. et al., “Identification of a 49-bp fragment of the HvLTP2 promoter directing aleruone cell specific expression” Gene 341:49-58 (2004). Such variants should retain promoter activity, particularly the ability to drive expression in seed or seed tissues. Activity can be measured by Northern blot analysis, reporter activity measurements when using transcriptional fusions, and the like. See, e.g., Sambrook et al. (1989) Molecular Cloning: A Laboratory Manual (2nd ed. Cold Spring Harbor Laboratory, Cold Spring Harbor, N.Y.), herein incorporated by reference.

Functional fragments can be obtained by use of restriction enzymes to cleave the naturally occurring regulatory element nucleotide sequences disclosed herein; by synthesizing a nucleotide sequence from the naturally occurring DNA sequence; or can be obtained through the use of PCR technology. See Mullis et al. (1987) Methods Enzymol. 155:335-350; Erlich, ed. (1989) PCR Technology (Stockton Press, New York).

For example, a routine way to remove part of a DNA sequence is to use an exonuclease in combination with DNA amplification to produce unidirectional nested deletions of double stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digests of a clone with this enzyme produces unidirectional nested deletions.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length, and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies. See, e.g., Innis et al. (1990) PCR Protocols, A Guide to Methods and Applications, eds., Academic Press).

The seed-preferred regulatory elements disclosed in the present invention, as well as variants and fragments thereof, are useful in the genetic manipulation of any plant when operably linked with an isolated nucleotide sequence of interest whose expression is to be controlled to achieve a desired phenotypic response.

By “operably linked” is intended a functional linkage between a promoter and a second sequence, wherein the promoter sequence initiates and mediates transcription of the DNA sequence corresponding to the second sequence. The expression cassette will include 5′ and 3′ regulatory sequences operably linked to at least one of the sequences of the invention.

In one typical embodiment, in the context of an expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

The regulatory elements of the invention can be operably linked to the isolated nucleotide sequence of interest in any of several ways known to one of skill in the art. The isolated nucleotide sequence of interest can be inserted into a site within the genome which is 3′ to the promoter of the invention using site specific integration as described in U.S. Pat. No. 6,187,994, incorporated by reference herein in its entirety.

The regulatory elements of the invention can be operably linked in expression cassettes along with isolated nucleotide sequences of interest for expression in the desired plant, more particularly in the seed of the plant. Such an expression cassette is provided with a plurality of restriction sites for insertion of the nucleotide sequence of interest under the transcriptional control of the regulatory elements.

The isolated nucleotides of interest expressed by the regulatory elements of the invention can be used for directing expression of a sequence in the seed or plant. This can be achieved by increasing expression of endogenous or exogenous products in seed. Alternatively, the results can be achieved by providing for a reduction of expression of one or more endogenous products, particularly enzymes or cofactors in the seed. This down regulation can be achieved through many different approaches known to one skilled in the art, including antisense, cosupression, use of hairpin formations, or others, and discussed infra. Importation or exportation of a cofactor also allows for control of seed composition. It is recognized that the regulatory elements may be used with their native or other coding sequences to increase or decrease expression of an operably linked sequence in the transformed plant or seed.

General categories of genes of interest for the purposes of the present invention include for example, those genes involved in information, such as zinc fingers; those involved in communication, such as kinases; and those involved in housekeeping, such as heat shock proteins. More specific categories of transgenes include genes encoding important traits for agronomics, insect resistance, disease resistance, herbicide resistance, and grain characteristics. Still other categories of transgenes include genes for inducing expression of exogenous products such as enzymes, cofactors, and hormones from plants and other eukaryotes as well as prokaryotic organisms.

Modifications that affect grain traits include increasing the content of oleic acid, or altering levels of saturated and unsaturated fatty acids. Likewise, the level of seed proteins, particularly modified seed proteins that improve the nutrient value of the seed, can be increased. This is achieved by the expression of such proteins having enhanced amino acid content.

Increasing the levels of lysine and sulfur-containing amino acids may be desired as well as the modification of starch type and content in the seed. Hordothionin protein modifications are described in WO 94/16078, filed Apr. 10, 1997; WO 96/38562, filed Mar. 26, 1997; WO 96/38563, filed Mar. 26, 1997; and U.S. Pat. No. 5,703,409, issued Dec. 30, 1997. Another example is lysine and/or sulfur-rich seed protein encoded by the soybean 2S albumin described in WO 97/35023, filed Mar. 20, 1996, and the chymotrypsin inhibitor from barley, Williamson et al. (1987) Eur. J. Biochem. 165:99-106.

Agronomic traits in seeds can be improved by altering expression of genes that: affect the response of seed or seed growth and development during environmental stress, Cheikh-N et al. (1994) Plant Physiol. 106(1):45-51) and genes controlling carbohydrate metabolism to reduce kernel abortion in maize, Zinselmeier et al. (1995) Plant Physiol. 107(2):385-391.

Modulation of nucleic acid expression (upregulating, downregulating, localizing, etc.) is therefore useful in many respects in plants. It is recognized that any nucleic acid of interest, including the native coding sequence, can be operably linked to the regulatory elements of the invention and expressed in the seed.

By way of illustration, without intending to be limiting, are examples of the types of genes which can be used in connection with the regulatory sequences of the invention.

1. Transgenes that provide improved output traits, including the following non-limiting examples:
(A) Altered fatty acids, for example, by
   (1) Down-regulation of stearoyl-ACP desaturase to increase stearic acid content of the plant. See Knultzon et al., Proc. Natl. Acad. Sci. USA 89: 2624 (1992) and WO99/64579 (Genes for Desaturases to Alter Lipid Profiles in Corn);
   (2) Elevating oleic acid via FAD-2 gene modification and/or decreasing linolenic acid via FAD-3 gene modification. See U.S. Pat. Nos. 6,063,947; 6,323,392; 6,372,965 and WO 93/11245);
   (3) Altering conjugated linolenic or linoleic acid content, such as in WO 01/12800;
   (4) Altering LEC1, AGP, Dek1, Superal1, mi1ps, various 1pa genes such as 1pa1, 1pa3, hpt or hggt. For example, see WO 02/42424, WO 98/22604, WO 03/011015, U.S. Pat. No. 6,423,886, U.S. Pat. No. 6,197,561, U.S. Pat. No. 6,825,397, US2003/0079247, US2003/0204870, WO02/057439, WO03/011015 and Rivera-Madrid, R. et. al. Proc. Natl. Acad. Sci. 92:5620-5624 (1995).
(B) Altered phosphorus content, for example, by the
   (1) Introduction of a phytase-encoding gene would enhance breakdown of phytate, adding more free phosphate to the transformed plant. See, e.g., Van Hartingsveldt et al., Gene 127: 87 (1993), for a disclosure of the nucleotide sequence of an Aspergillus niger phytase gene.
   (2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy et al., Maydica 35: 383 (1990) and/or by altering inositol kinase activity as in WO 02/059324, US2003/0009011, WO 03/027243, US2003/0079247, WO 99/05298, U.S. Pat. No. 6,197,561, U.S. Pat. No. 6,291,224, U.S. Pat. No. 6,391,348, WO2002/059324, US2003/0079247, WO98/45448, WO99/55882, WO01/04147.
(C) Altered carbohydrates effected, for example, by altering a gene for an enzyme that affects the branching pattern of starch or a gene altering thioredoxin. See U.S. Pat. No. 6,531,648; see also Shiroza et al., J. Bacteriol. 170: 810 (1988) (nucleotide sequence of Streptococcus mutans fructosyltransferase gene); Steinmetz et al., Mol. Gen. Genet. 200: 220 (1985) (nucleotide sequence of Bacillus subtilis levansucrase gene); Pen et al., Bio/Technology 10: 292 (1992) (production of transgenic plants that express Bacillus licheniformis alpha-amylase); Elliot et al., Plant Molec. Biol. 21: 515 (1993) (nucleotide sequences of tomato invertase genes); Søgaard et al., J. Biol. Chem. 268: 22480 (1993) (site-directed mutagenesis of barley alpha-amylase gene); Fisher et al., Plant Physiol. 102: 1045 (1993) (maize endosperm starch branching enzyme II); WO 99/10498 (improved digestibility and/or starch extraction through modification of UDP-D-xylose 4-epimerase, Fragile 1 and 2, Ref1, HCHL, C4H); U.S. Pat. No. 6,232,529 (method of producing high oil seed by modification of starch levels (AGP)). The fatty acid modification genes mentioned above may also be used to affect starch content and/or composition through the interrelationship of the starch and oil pathways.
(D) Altered antioxidant content or composition, such as alteration of tocopherol or tocotrienols. See, e.g., U.S. Pat. No. 6,787,683, US2004/0034886 and WO 00/68393 involving the manipulation of antioxidant levels through alteration of a phytl prenyl transferase (ppt), WO 03/082899 through alteration of a homogentisate geranylgeranyl transferase (hggt).
(E) Altered essential seed amino acids. See, e.g., U.S. Pat. No. 6,127,600 (method of increasing accumulation of essential amino acids in seeds); U.S. Pat. No. 6,080,913 (binary methods of increasing accumulation of essential amino acids in seeds); U.S. Pat. No. 5,990,389 (high lysine); U.S. Pat. No. 5,850,016 (alteration of amino acid compositions in seeds); WO98/20133 (proteins with enhanced levels of essential amino acids); U.S. Pat. No. 5,885,802 (high methionine); U.S. Pat. No. 5,885,801 (high threonine); U.S. Pat. No. 6,664,445 (plant amino acid biosynthetic enzymes), U.S. Pat. No. 6,459,019 (increased lysine and threonine); U.S. Pat. No. 6,441,274 (plant tryptophan synthase beta subunit); U.S. Pat. No. 6,346,403 (methionine metabolic enzymes); U.S. Pat. No. 5,939,599 (high sulfur); U.S. Pat. No. 5,912,414 (increased methionine); U.S. Pat. No. 5,633,436 (increasing sulfur amino acid content); U.S. Pat. No. 5,559,223 (synthetic storage proteins with defined structure containing programmable levels of essential amino acids for improvement of the nutritional value of plants); U.S. Pat. No. 6,194,638 (hemicellulose); U.S. Pat. No. 6,194,638 (RGP); U.S. Pat. No. 6,399,859 and US2004/0025203 (UDPGdH); US2003/0163838, US2003/0150014, US2004/0068767, 6,803,498, WO01/79516, and WO00/09706 (Ces A: cellulose synthase); WO98/56935 (plant amino acid biosynthetic enzymes); WO98/45458 (engineered seed protein having higher percentage of essential amino acids); WO98/42831 (increased lysine); WO96/01905 (increased threonine); WO95/15392 (increased lysine); WO99/40209 (alteration of amino acid compositions in seeds); WO99/29882 (methods for altering amino acid content of proteins).

2. Transgenes that provide improved agronomic traits such as the following non-limiting examples:

Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. See, e.g., WO 00/73475 (water use efficiency is altered through alteration of malate); U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, WO2000/060089, WO2001/026459, WO2001/035725, WO2001/034726, WO2001/035727, WO2001/036444, WO2001/036597, WO2001/036598, WO2002/015675, WO2002/017430, WO2002/077185, WO2002/079403, WO2003/013227, WO2003/013228, WO2003/014327, WO2004/031349, WO2004/076638, WO98/09521, and WO99/38977 describing genes, including CBF genes and transcription factors effective in mitigating the negative effects of freezing, high salinity, and drought on plants, as well as conferring other positive effects on plant phenotype; US2004/0148654 and WO01/36596 where abscisic acid is altered in plants resulting in improved plant phenotype such as increased yield and/or increased tolerance to abiotic stress; WO2000/006341, WO04/090143, U.S. application Ser. Nos. 10/817483 and 09/545,334 where cytokinin expression is modified resulting in plants with increased stress tolerance, such as drought tolerance, and/or increased yield; WO02/02776, WO2003/052063, JP2002281975, U.S. Pat. No. 6,084,153, WO0164898, U.S. Pat. No. 6,177,275, and U.S. Pat. No. 6,107,547 (enhancement of nitrogen utilization and altered nitrogen responsiveness); US20040128719, US20030166197 and WO2000/32761 (ethylene alteration); US20040098764 and US20040078852 (plant transcription factors or transcriptional regulators of abiotic stress).

Other genes and transcription factors that affect plant growth and agronomic traits such as yield, flowering, plant growth and/or plant structure, can be introduced or introgressed into plants. See, e.g., WO97/49811 (LHY); WO98/56918 (ESD4); WO97/10339 and U.S. Pat. No. 6,573,430 (TFL); U.S. Pat. No. 6,713,663 (FT); WO96/14414 (CON); WO96/38560, WO01/21822 (VRN1); WO00/44918 (VRN2); WO99/49064 (GI); WO00/46358 (FRI); WO97/29123, U.S. Pat. No. 6,794,560, U.S. Pat. No. 6,307,126 (GAI); WO99/09174 (D8 and Rht); WO2004/076638 and WO2004/031349 (transcription factors). Commercial traits in plants can be created through the expression of genes that alter starch or protein for the production of paper, textiles, ethanol, polymers or other materials with industrial uses.

3. Genes that Control Male-sterility

There are several methods of conferring genetic male sterility available, such as multiple mutant genes at separate locations within the genome that confer male sterility, as disclosed in U.S. Pat. Nos. 4,654,465 and 4,727,219 to Brar et al. and chromosomal translocations as described by Patterson in U.S. Pat. Nos. 3,861,709 and 3,710,511. In addition to these methods, Albertsen et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on”, the promoter, which in turn allows the gene that confers male fertility to be transcribed.

(A) Introduction of a deacetylase gene under the control of a tapetum-specific promoter and with the application of the chemical N-Ac-PPT. See WO 01/29237.

(B) Introduction of various stamen-specific promoters. See WO 92/13956, WO 92/13957.

(C) Introduction of the barnase and the barstar gene. See Paul et al. Plant Mol. Biol. 19:611-622, 1992.

For additional examples of nuclear male and female sterility systems and genes, see also U.S. Pat. No. 5,859,341; U.S. Pat. No. 6,297,426; U.S. Pat. No. 5,478,369; U.S. Pat. No. 5,824,524; U.S. Pat. No. 5,850,014; and U.S. Pat. No. 6,265,640.

4. Genes that create a site for site specific DNA integration. This includes the introduction of FRT sites that may be used in the FLP/FRT system and/or Lox sites that may be used in the Cre/Loxp system. See, e.g., Lyznik, et al., Site-Specific Recombination for Genetic Engineering in Plants, Plant Cell Rep (2003) 21:925-932; WO 99/25821, which are hereby incorporated by reference. Other systems that may be used include the Gin recombinase of phage Mu (Maeser et al., 1991, Mol Gen Genet.;230(1-2):170-6.); Vicki Chandler, The Maize Handbook ch. 118 (Springer-Verlag 1994), the Pin recombinase of E. coli (Enomoto et al., 1983), and the R/RS system of the pSR1 plasmid (Araki et al., 1992. J Mol Biol. 5;225(1):25-37.

Means of increasing or inhibiting a protein are well known to one skilled in the art and, by way of example, may include, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING (Targeting Induced Local Lesions In Genomes), and biosynthetic competition to manipulate, the expression of proteins.

Many techniques for gene silencing are well known to one of skill in the art, including but not limited to knock-outs (such as by insertion of a transposable element such as Mu, Vicki Chandler, The Maize Handbook ch. 118 (Springer-Verlag 1994) or other genetic elements such as a FRT, Lox or other site specific integration site; RNA interference (Napoli et al. (1990) Plant Cell 2:279-289; U.S. Pat. No. 5,034,323, Sharp (1999) Genes Dev. 13:139-141, Zamore et al. (2000) Cell 101:25-33; and Montgomery et al. (1998) PNAS USA 95:15502-15507); virus-induced gene silencing (Burton, et al. (2000) Plant Cell 12:691-705, and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman & Sakai (2003) Plant Cell 15:2730-2741); ribozymes (Steinecke et al. (1992) EMBO J. 11:1525, and Perriman et al. (1993) Antisense Res. Dev. 3:253); oligonucleotide mediated targeted modification (e.g., WO 03/076574 and WO 99/25853); zinc-finger targeted molecules (e.g., WO 01/52620; WO 03/048345; and WO 00/42219); and other methods or combinations of the above methods known to those of skill in the art.

Any method of increasing or inhibiting a protein can be used in the present invention. Several examples are outlined in more detail below for illustrative purposes.

The nucleotide sequence operably linked to the regulatory elements disclosed herein can be an antisense sequence for a targeted gene. See, e.g., Sheehy et al. (1988) PNAS USA 85:8805-8809; U.S. Pat. Nos. 5,107,065; 5,453, 566; 5,759,829. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences disclosed herein can be operably linked to antisense DNA sequences to reduce or inhibit expression of a native protein in the plant seed.

As noted, other potential approaches to impact expression of proteins in the seed include traditional co-suppression, that is, inhibition of expression of an endogenous gene through the expression of an identical structural gene or gene fragment introduced through transformation (Goring, D. R., Thomson, L., Rothstein, S. J. 1991. Proc. Natl. Acad Sci. USA 88:1770-1774 co-suppression; Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12: 883-888; and Neuhuber et al. (1994) Mol. Gen. Genet. 244:230-241). In one example, co-suppression can be achieved by linking the promoter to a DNA segment such that transcripts of the segment are produced in the sense orientation and where the transcripts have at least 65% sequence identity to transcripts of the endogenous gene of interest, thereby suppressing expression of the endogenous gene in said plant cell. See U.S. Pat. No. 5,283,184. The endogenous gene targeted for co-suppression may be a gene encoding any protein that accumulates in the plant species of interest. For example, where the endogenous gene targeted for co-suppression is the 50 kD gamma-zein gene, co-suppression is achieved using an expression cassette comprising the 50 kD gamma-zein gene sequence, or variant or fragment thereof.

Additional methods of co-suppression are known in the art and can be similarly applied to the instant invention. These methods involve the silencing of a targeted gene by spliced hairpin RNA's and similar methods also called RNA interference and promoter silencing. See Smith et al. (2000) Nature 407:319-320; Waterhouse and Helliwell (2003)) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Plant Phystiol. 129:1723-1731; Patent Applications WO 99/53050; WO 99/49029; WO 99/61631; WO 00/49035; U.S. Pat. No. 6,506,559.

For mRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). miRNA molecules are highly efficient at inhibiting the expression of endogenous genes, and the RNA interference they induce is inherited by subsequent generations of plants.

In one embodiment, the polynucleotide to be introduced into the plant comprises an inhibitory sequence that encodes a zinc finger protein that binds to a gene encoding a protein of the invention resulting in reduced expression of the gene. In particular embodiments, the zinc finger protein binds to a regulatory region of a gene of the invention. In other embodiments, the zinc finger protein binds to a messenger RNA encoding a protein and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 20030037355.

The expression cassette may also include at the 3′ terminus of the isolated nucleotide sequence of interest, a transcriptional and translational termination region functional in plants. The termination region can be native with the promoter nucleotide sequence of the present invention, can be native with the DNA sequence of interest, or can be derived from another source.

The ZM-EAP2 terminator set forth in SEQ ID NO:2 is 531 nucleotides in length. The coding region was identified according to the procedure described in Woo et al, Journal Plant Cell 13(10), 2297-2317 (2001) incorporated herein by reference. The terminator, with the appropriate promoter, can provide expression during about 20-40 DAP development. The ZM-EAP2 terminator can be used with the ZM-EAP2 promoter in an expression cassette, or can be used with another appropriate promoter to provide seed-preferred expression of a coding region.

Any convenient termination regions can be used in conjunction with the promoter of the invention, and are available from the Ti-plasmid of A. tumefaciens, such as the octopine synthase and nopaline synthase termination regions. See Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The expression cassettes can additionally contain 5′ leader sequences. Such leader sequences can act to enhance translation. Translation leaders are known in the art and include: picomavirus leaders, for example, EMCV leader (Encephalomyocarditis 5′ noncoding region), Elroy-Stein et al. (1989) Proc. Natl. Acad. Sci. USA 86:6126-6130; potyvirus leaders, for example, TEV leader (Tobacco Etch Virus), Allison et al. (1986); MDMV leader (Maize Dwarf Mosaic Virus), Virology 154:9-20; human immunoglobulin heavy-chain binding protein (BiP), Macejak et al. (1991) Nature 353:90-94; untranslated leader from the coat protein mRNA of alfalfa mosaic virus (AMV RNA 4), Jobling et al. (1987) Nature 325:622-625); tobacco mosaic virus leader (TMV), Gallie et al. (1989) Molecular Biology of RNA, pages 237-256; and maize chlorotic mottle virus leader (MCMV), Lommel et al. (1991) Virology 81:382-385. See also Della-Cioppa et al. (1987) Plant Physiology 84:965-968. The cassette can also contain sequences that enhance translation and/or mRNA stability such as introns.

In those instances where it is desirable to have an expressed product of an isolated nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to: the transit peptide for the acyl carrier protein, the small subunit of RUBISCO, plant EPSP synthase, and the like.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions such as transitions and transversions, can be involved.

As noted herein, the present invention provides vectors capable of expressing genes of interest under the control of the regulatory elements. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook et al. (supra).

The transformation vector comprising the regulatory sequences of the present invention operably linked to an isolated nucleotide sequence in an expression cassette, can also contain at least one additional nucleotide sequence for a gene to be cotransformed into the organism. Alternatively, the additional sequence(s) can be provided on another transformation vector.

Vectors that are functional in plants can be binary plasmids derived from Agrobacterium. Such vectors are capable of transforming plant cells. These vectors contain left and right border sequences that are required for integration into the host (plant) chromosome. At minimum, between these border sequences is the gene to be expressed under control of the regulatory elements of the present invention. In one embodiment, a selectable marker and a reporter gene are also included. For ease of obtaining sufficient quantities of vector, a bacterial origin that allows replication in E. coli can be used.

Reporter genes can be included in the transformation vectors. Examples of suitable reporter genes known in the art can be found in, for example: Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; Kain et al. (1995) BioTechniques 19:650-655; and Chiu et al. (1996) Current Biology 6:325-330.

Selectable marker genes for selection of transformed cells or tissues can be included in the transformation vectors. These can include genes that confer antibiotic resistance or resistance to herbicides. Examples of suitable selectable marker genes include, but are not limited to: genes encoding resistance to chloramphenicol, Herrera Estrella et al. (1983) EMBO J. 2:987-992; methotrexate, Herrera Estrella et al. (1983) Nature 303:209-213; Meijer et al. (1991) Plant Mol. Biol. 16:807-820; hygromycin, Waldron et al. (1985) Plant Mol. Biol. 5:103-108; Zhijian et al. (1995) Plant Science 108:219-227; streptomycin, Jones et al. (1987) Mol. Gen. Genet. 210:86-91; spectinomycin, Bretagne-Sagnard et al. (1996) Transgenic Res. 5:131-137; bleomycin, Hille et al. (1990) Plant Mol. Biol. 7:171-176; sulfonamide, Guerineau et al. (1990) Plant Mol. Biol. 15:127-136; bromoxynil, Stalker et al. (1988) Science 242:419-423; glyphosate, Shaw et al. (1986) Science 233:478-481; phosphinothricin, DeBlock et al. (1987) EMBO J. 6:2513-2518.

Further, when linking a seed promoter of the invention with a nucleotide sequence encoding a detectable protein, expression of a linked sequence can be tracked in the seed, thereby providing a useful so-called screenable or scorable markers. The expression of the linked protein can be detected without the necessity of destroying tissue. More recently, interest has increased in utilization of screenable or scorable markers. By way of example without limitation, the promoter can be linked with detectable markers including a β-glucuronidase, or uidA gene (GUS), which encodes an enzyme for which various chromogenic substrates are known (Jefferson, R. A. et al., 1986, Proc. Natl. Acad. Sci. USA 83:8447-8451); maize-optimized phosphinothricin acetyl transferase (moPAT); chloramphenicol acetyl transferase; alkaline phosphatase; a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988); Ludwig et al. (1990) Science 247:449); a p-lactamase gene (Sutcliffe, Proc. Nat'l. Acad. Sci. U.S.A. 75:3737 (1978)), which encodes an enzyme for which various chromogenic substrates are known (e.g., PADAC, a chromogenic cephalosporin); a xylE gene (Zukowsky et al., Proc. Nat'l. Acad. Sci. U.S.A. 80:1101 (1983)), which encodes a catechol dioxygenase that can convert chromogenic catechols; an α-amylase gene (Ikuta et al., Biotech. 8:241 (1990)); a tyrosinase gene (Katz et al., J. Gen. Microbiol. 129:2703 (1983)), which encodes an enzyme capable of oxidizing tyrosine to DOPA and dopaquinone, which in turn condenses to form the easily detectable compound melanin a green fluorescent protein (GFP) gene (Sheen et al., Plant J. 8(5):777-84 (1995)); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); DS-RED EXPRESS (Matz, M. V. et al (1999) Nature Biotech. 17:969-973, Bevis B. J et al. (2002) Nature Biotech 20:83-87, Haas, J. et al. (1996) Curr. Biol. 6:315-324); Zoanthus sp. yellow fluorescent protein (ZsYellow) that has been engineered for brighter fluorescence (Matz et al. (1999) Nature Biotech. 17:969-973, available from BD Biosciences Clontech, Palo Alto, Calif., USA, catalog no. K6100-1); and cyan florescent protein (CYP) (Bolte et al. (2004) J. Cell Science 117:943-54 and Kato et al. (2002) Plant Physiol 129:913-42).

A transformation vector comprising the particular regulatory sequences of the present invention, operably linked to an isolated nucleotide sequence of interest in an expression cassette, can be used to transform any plant. In this manner, genetically modified plants, plant cells, plant tissue, seed, and the like can be obtained. Transformation protocols can vary depending on the type of plant or plant cell, i.e., monocot or dicot, targeted for transformation. Suitable methods of transforming plant cells include microinjection, Crossway et al. (1986) Biotechniques 4:320-334; electroporation, Riggs et al. (1986) Proc. Natl. Acad. Sci. USA 83:5602-5606; Agrobacterium-mediated transformation, see for example, Townsend et al. U.S. Pat. No. 5,563,055; direct gene transfer, Paszkowski et al. (1984) EMBO J. 3:2717-2722; and ballistic particle acceleration, see for example, Sanford et al. U.S. Pat. No. 4,945,050, Tomes et al. (1995) in Plant Cell, Tissue, and Organ Culture: Fundamental Methods, ed. Gamborg and Phillips (Springer-Verlag, Berlin); and McCabe et al. (1988) Biotechnology 6:923-926; see also Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Datta et al. (1990) Bio/Technology 8:736-740 (rice); Klein et al. (1988) Proc. Natl. Acad. Sci. USA 85:4305-4309 (maize); Klein et al. (1988) Biotechnology 6:559-563 (maize); Klein et al. (1988) Plant Physiol. 91:440-444 (maize); Fromm et al. (1990) Biotechnology 8:833-839; Hooydaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. G. P. Chapman et al. (Longman, N.Y.), pp. 197-209 (pollen); Kaeppler et al. (1990) Plant Cell Reports 9:415-418; and Kaeppler et al. (1992) Theor. Appl. Genet. 84:560-566 (whisker-mediated transformation); D. Halluin et al. (1992) Plant Cell 4:1495-1505 (electroporation); Li et al. (1993) Plant Cell Reports 12:250-255 and Christou et al. (1995) Annals of Botany 75:407-413 (rice); Osjoda et al. (1996) Nature Biotechnology 14:745-750 (maize via Agrobacterium tumefaciens).

The cells that have been transformed can be grown into plants in accordance with conventional methods. See, for example, McCormick et al. (1986) Plant Cell Reports 5:81-84. These plants can then be grown and pollinated with the same transformed strain or different strains. The resulting plant having seed-preferred expression of the desired phenotypic characteristic can then be identified. Two or more generations can be grown to ensure that seed-preferred expression of the desired phenotypic characteristic is stably maintained and inherited.

The following examples are offered by way of illustration and not by way of limitation.

### EXAMPLES

Regulatory regions from maize EAP2 were isolated from maize plants and cloned. Maize EAP2 was selected as a source of seed-preferred regulatory elements based on the spatial and temporal expression of its products.

### Example 1

### Prediction of Expression via Lynx MPSS

Lynx™ gene expression profiling technology was used to identify the maize EAP2 coding region as a candidate for promoter isolation. Massively parallel signature sequencing (MPSS, see Brenner et al, Nature Biotechnology 18:630-634, 2000) indicated expression in various genotypes at about 20 DAP (days after pollination) in seed, peaking at about 18 k ppm. Results are summarized in FIG. 1. MPSS data showed no significant expression of maize EAP2 in flowering or vegetative tissue.

### Example 2

### Expression Data Using Promoter Sequences

A promoter::DS-RED EXPRESS::terminator fusion construct was prepared as set out below. DS-RED EXPRESS is a scorable marker (Matz, M. V. et al 91999) Nature Biotech. 17:969-973, Bevis B. J et al. (2002) Nature Biotech 20:83-87, Haas, J. et al. (1996) Curr. Biol. 6:315-324). All vectors were constructed using standard molecular biology techniques (Sambrook et al., supra). The fusion construct was constructed as follows: ZM-EAP2 PRO (SEQ ID NO: 1):DS-RED EXPRESS:ZM-EAP2 TERM.

Successful subcloning was confirmed by restriction analysis. Transformation and expression was confirmed as discussed infra.

### Example 3

### Confirmation of Expression

The construct described above was inserted into the genome of various zea mays plants through techniques well-known in the art. Seed was collected at various numbers of days after pollination (DAP). The collected seed was bisected and examined under a microscope with the appropriate filter to detect the expression of the DS-RED EXPRESS marker. Photographs were taken of seeds at 17 DAP, 22 DAP, 25 DAP, 30 DAP, and 33 DAP, showing preferred expression of the DS-RED EXPRESS marker in the embryo and aleurone. The results of these tests are shown in FIGS. 2 and 3.

### Example 4

### Expression Using Truncated Promoter Sequence

In addition to the testing of the full EAP2 promoter described in Examples 1-3 above, similar tests were conducted on a truncation of the EAP2 promoter, designated the EAP2B sequence. Similar to the procedure described in Example 2, a fusion construct was produced as follows: ZM-EAP2B PRO (SEQ ID NO:4):DS-RED EXPRESS:ZM-EAP2 TERM. Photographs were taken of seeds at 21 DAP and 25 DAP, showing preferred expression of the DS-RED EXPRESS marker in the embryo and aleurone, similar to the results seen with the full EAP2 promoter described in Example 3 supra. The results of these tests are shown in FIG. 4.

Although the foregoing invention has been described in some detail by way of illustration and example for purposes of clarity of understanding, it will be obvious that certain changes and modifications may be practiced within the scope of the appended claims. All references cited herein are incorporated by reference in their entirety.

What is claimed is:

1. An isolated regulatory sequence that regulates transcription in a seed embryo-preferred manner, wherein the regulatory sequence comprises a nucleotide selected from the group consisting of:
(a) sequences natively associated with, and that drive expression in, the coding regions of maize eap2 (embryo abundant protein 2);
(b) the nucleotide sequence of SEQ ID NO:1;
(c) a functional fragment of the nucleotide sequence of SEQ ID NO:1;
(d) the nucleotide sequence of SEQ ID NO:2; and
(e) the nucleotide sequence of SEQ ID NO:4.

2. A recombinant expression cassette comprising the regulatory sequence of claim 1 operably linked to a nucleic acid of interest.

3. A vector comprising the recombinant expression cassette of claim 2.

4. A plant cell having stably incorporated in its genome the isolated regulatory sequence of claim 1.

5. The plant cell of claim 4, wherein the plant is a monocot or dicot.

6. The plant of claim 5, wherein the plant is maize, wheat, rice, millet, barley, sorghum, rye, soybean, alfalfa, oilseed Brassica, cotton, sunflower, potato, or tomato.

7. A plant stably transformed with the isolated regulatory sequence of claim 1.

8. Transgenic seed of the plant of claim 7, wherein the seed comprises the isolated regulatory sequence of claim 1.

9. A method for modulating expression of a nucleic acid of interest in a plant, said method comprising:
a) introducing into a plant cell or tissue a polynucleotide molecule comprising the regulatory sequence of claim 1 operably linked to a nucleic acid of interest; and
b) regenerating a plant from the plant cell wherein expression of the nucleic acid is modulated compared to the wild-type.

10. The method of claim 9, wherein the nucleic acid of interest is selected from the group consisting of a nucleic acids providing improved output traits, nucleic acids providing improved agronomic traits, nucleic acids providing resistance to insects, nucleic acids providing resistance to disease and nucleic acids providing herbicide resistance.

11. The method of claim 10, wherein the nucleic acid of interest is a nucleic acid providing improved output traits.
